(19) United States
(12) Patent Application Publication
(10) Pub. No.: US 2010/0331640 A1
(43) Pub. Date: Dec. 30, 2010

(54) USE OF PHOTODETECTOR ARRAY TO IMPROVE EFFICIENCY AND ACCURACY OF AN OPTICAL MEDICAL SENSOR

(75) Inventor: Casey V. Medina, Westminster, CO (US)

Correspondence Address: NELLCOR PURITAN BENNETT LLC;ATTN: IP LEGAL, 6135 Gunbarrel Avenue, Boulder, CO, 80301, US

(73) Assignee: Nellcor Puritan Bennett LLC, Boulder, CO (US)

(21) Appl. No.: 12/492,377

(22) Filed: Jun. 26, 2009

(51) Int. Cl. A61B 5/1455

(52) U.S. Cl. 600/324

(57) ABSTRACT

A system and method for determining physiological parameters of a patient based on light transmitted through the patient. The light may be transmitted via an emitter and received by a detector array that includes a plurality of detector elements. The emitter and the detector may both be located on a flexible substrate.

### BACKGROUND

The present disclosure relates generally to medical devices and, more particularly, to sensors used for sensing physiological parameters of a patient.

This section is intended to introduce the reader to various aspects of all that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

In the field of medicine, doctors often desire to monitor certain physiological characteristics of their patients. Accordingly, a wide variety of devices have been developed for monitoring many such physiological characteristics. Such devices provide doctors and other healthcare personnel with the information they need to provide the best possible healthcare for their patients. As a result, such monitoring devices have become an indispensable part of modern medicine.

One technique for monitoring certain physiological characteristics of a patient is commonly referred to as pulse oximetry, and the devices built based upon pulse oximetry techniques are commonly referred to as pulse oximeters. Pulse oximetiy may be used to measure various blood flow characteristics, such as the blood-oxygen saturation of hemoglobin in arterial blood, the volume of individual blood pulsations supplying the tissue, and/or the rate of blood pulsations corresponding to each heartbeat of a patient. In fact, the “pulse” in pulse oximetry refers to the time varying amount of arterial blood in the tissue during each cardiac cycle.

Pulse oximeters typically utilize a non-invasive sensor that transmits light through a patient's tissue and that photoelectrically detects the absorption and/or scattering of the transmitted light in such tissue. One or more of the above physiological characteristics may then be calculated based upon the amount of light absorbed or scattered. More specifically, the light passed through the tissue is typically selected to be of one or more wavelengths that may be absorbed or scattered by the blood in an amount correlative to the amount of the blood constituent present in the blood. The amount of light absorbed and/or scattered may then be used to estimate the amount of blood constituent in the tissue using various algorithms.

The light sources utilized in pulse oximeters typically are placed in a certain position on a patient. For the sensor to operate properly, this position must be maintained. Accordingly, movement of the sensor due to the movements of a patient, may lead to signal noise.

### BRIEF DESCRIPTION OF THE DRAWINGS

Advantages of the disclosed techniques may become apparent upon reading the following detailed description and upon reference to the drawings in which:

FIG. 1 illustrates a perspective view of a pulse oximeter in accordance with an embodiment;

FIG. 2 illustrates a simplified block diagram of a pulse oximeter in FIG. 1, according to an embodiment;

FIG. 3 illustrates a top view of a sensor of FIG. 2, according to an embodiment;

FIG. 4 illustrates a side view of the sensor of FIG. 3, according an embodiment;

FIG. 5 illustrates a top view of a sensor of FIG. 2, according to a second embodiment; and

FIG. 6 illustrates a side view of the sensor of FIG. 5, according to the second embodiment.

### DETAILED DESCRIPTION OF SPECIFIC EMBODIMENTS

One or more specific embodiments of the present disclosure will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Present embodiments relate to non-invasively measuring physiologic parameters corresponding to blood flow in a patient by emitting light into a patient's tissue with light emitters (e.g., light emitting diodes) and photoelectrically detecting the light after it has passed through the patient's tissue. More specifically, present embodiments are directed to increasing the effective area of photodetectors in a pulse oximetry sensor. Utilization of a photodetector array made up of a plurality of photodetectors may allow for increased efficiency of the overall pulse oximetry system by being able to receive signals at more than one location. Thus, if a path between an emitter and a detector is blocked by tissue, bone, or other constituents, a secondary path between the emitter and a second detector may be used to transmit light signals. Also, a photodetector array may be scanned to determine which individual detectors in the array are receiving the strongest light transmission from an emitter. This detector may then be chosen and signals received from this detector may then be utilized to calculate physiological parameters of a patient. The detector array may also be placed on a flexible substrate so as to allow the sensor to be more form fitting.

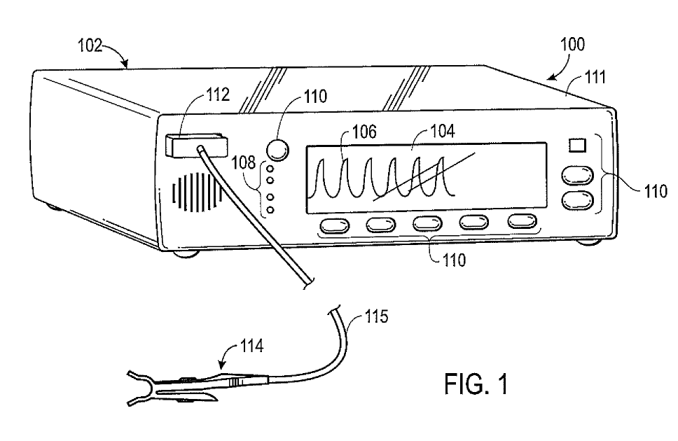

Turning to FIG. 1, a perspective view of a medical device is illustrated in accordance with an embodiment. The medical device may be a pulse oximeter 100. The pulse oximeter 100 may include a monitor 102, such as those available from Nellcor Puritan Bennett LLC. The monitor 102 may be configured to display calculated parameters on a display 104. As illustrated in FIG. 1, the display 104 may be integrated into the monitor 102. However, the monitor 102 may be configured to provide data via a port to a display (not shown) that is not integrated with the monitor 102. The display 104 may be configured to display computed physiological data including, for example, an oxygen saturation percentage, a pulse rate, and/or a plethysmographic waveform 106. As is known in the art, the oxygen saturation percentage may be a functional arterial hemoglobin oxygen saturation measurement in units of percentage SpO₂, while the pulse rate may indicate a patient's pulse rate in beats per minute. The monitor 102 may also display information related to alarms, monitor settings, and/or signal quality via indicator lights 108.

To facilitate user input, the monitor 102 may include a plurality of control inputs 110. The control inputs 110 may include fixed function keys, programmable function keys, and soft keys. Specifically, the control inputs 110 may correspond to soft key icons in the display 104. Pressing control inputs 110 associated with, or adjacent to, an icon in the display may select a corresponding option. The monitor 102 may also include a casing 111. The casing 111 may aid in the protection of the internal elements of the monitor 102 from damage.

The monitor 102 may further include a sensor port 112. The sensor port 112 may allow for connection to an external sensor 114, via a cable 115 which connects to the sensor port 112. Alternatively, the external sensor 114 may be wirelessly coupled the monitor 102. Furthermore, the sensor 114 may be of a disposable or a non-disposable type. The sensor 114 may obtain readings from a patient, which can be used by the monitor to calculate certain physiological characteristics such as the blood-oxygen saturation of hemoglobin in arterial blood, the volume of individual blood pulsations supplying the tissue, and/or the rate of blood pulsations corresponding to each heartbeat of a patient.

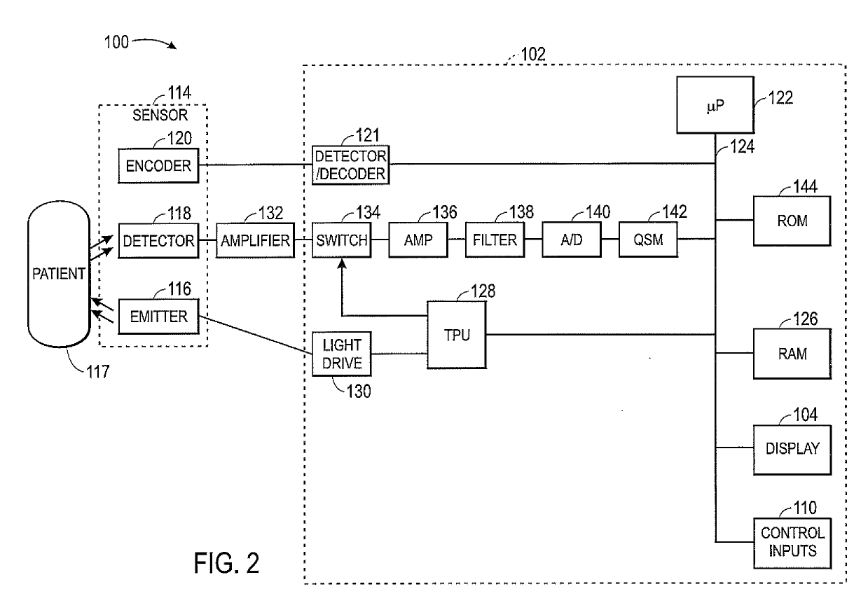

Turning to FIG. 2, a simplified block diagram of a pulse oximeter 100 is illustrated in accordance with an embodiment. Specifically, certain components of the sensor 114 and the monitor 102 are illustrated in FIG. 2. The sensor 114 may include an emitter 116, a detector 118, and an encoder 120. It should be noted that the emitter 116 may be capable of emitting at least two wavelengths of light, e.g., RED and infrared (IR) light, into the tissue of a patient 117 to calculate the patient's 117 physiological characteristics, where the RED wavelength may be between about 600 nanometers (nm) and about 700 nm, and the IR wavelength may be between about 800 nm and about 1000 nm. The emitter 116 may include a single emitting device, for example, with two light emitting diodes (LEDs) or the emitter 116 may include a plurality of emitting devices with, for example, multiple LED's at various locations. Regardless of the number of emitting devices, the emitter 116 may be used to measure, for example, water fractions, hematocrit, or other physiologic parameters of the patient 117. It should be understood that, as used herein, the term “light” may refer to one or more of ultrasound, radio, microwave, millimeter wave, infrared, visible, ultraviolet, gamma ray or X-ray electromagnetic radiation, and may also include any wavelength within the radio, microwave, infrared, visible, ultraviolet, or X-ray spectra, and that any suitable wavelength of light may be appropriate for use with the present disclosure.

In one embodiment, the detector 118 may be an array of detector elements that may be capable of detecting light at various intensities and wavelengths. In operation, light enters the detector 118 after passing through the tissue of the patient 117. The detector 118 may convert the light at a given intensity, which may be directly related to the absorbance and/or reflectance of light in the tissue of the patient 117, into an electrical signal. That is, when more light at a certain wavelength is absorbed or reflected, less light of that wavelength is typically received from the tissue by the detector 118. After converting the received light to an electrical signal, the detector 118 may send the signal to the monitor 102, where physiological characteristics may be calculated based at least in part on the absorption of light in the tissue of the patient 117.

Additionally the sensor 114 may include an encoder 120, which may contain information about the sensor 114, such as what type of sensor it is (e.g., whether the sensor is intended for placement on a forehead or digit) and the wavelengths of light emitted by the emitter 116. This information may allow the monitor 102 to select appropriate algorithms and/or calibration coefficients for calculating the patient's 117 physiological characteristics. The encoder 120 may, for instance, be a memory on which one or more of the following information may be stored for communication to the monitor 102: the type of the sensor 114; the wavelengths of light emitted by the emitter 116; and the proper calibration coefficients and/or algorithms to be used for calculating the patient's 117 physiological characteristics. In one embodiment, the data or signal from the encoder 120 may be decoded by a detector/decoder 121 in the monitor 102.

Signals from the detector 118 and the encoder 120 may be transmitted to the monitor 102. The monitor 102 may include one or more processors 122 coupled to an internal bus 124. Also connected to the bus may be a RAM memory 126 and a display 104. A time processing unit (TPU) 128 may provide timing control signals to light drive circuitry 130, which controls when the emitter 116 is activated, and if multiple light sources are used, the multiplexed timing for the different light sources. TPU 128 may also control the gating-in of signals from detector 118 through an amplifier 132 and a switching circuit 134. These signals are sampled at the proper time, depending at least in part upon which of multiple light sources is activated, if multiple light sources are used. The received signal from the detector 118 may be passed through an amplifier 136, a low pass filter 138, and an analog-to-digital converter 140 for amplifying, filtering, and digitizing the electrical signals the from the sensor 114. The digital data may then be stored in a queued serial module (QSM) 142, for later downloading to RAM 126 as QSM 142 fills up. In an embodiment, there may be multiple parallel paths for separate amplifiers, filters, and A/D converters for multiple light wavelengths or spectra received.

In an embodiment, based at least in part upon the received signals corresponding to the light received by detector 118, processor 122 may calculate the oxygen saturation using various algorithms. These algorithms may require coefficients, which may be empirically determined. For example, algorithms relating to the distance between an emitter 116 and various detector elements in a detector 118 may be stored in a ROM 144 and accessed and operated according to processor 122 instructions. The processor 122 may also be utilized to scan for a particular signal from a detector element in a detector array of the detector 118, as will be described in greater detail below.

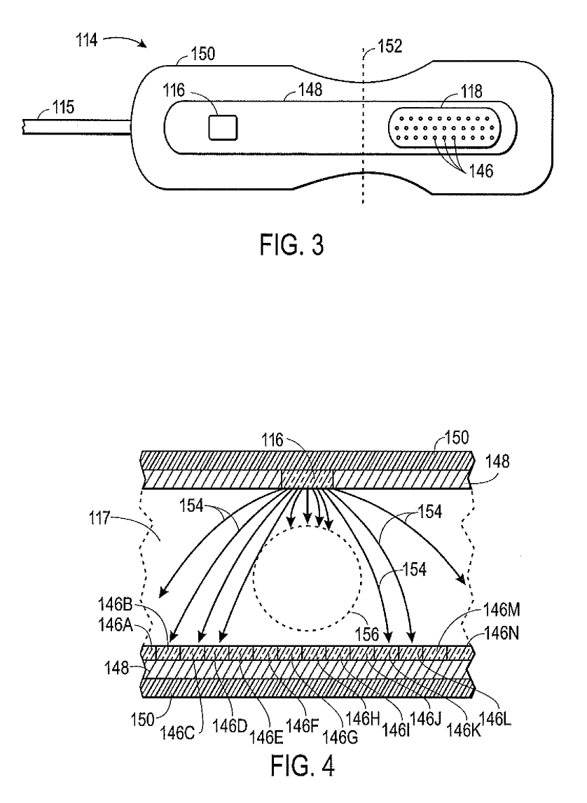

FIG. 3 illustrates an embodiment of the sensor 114 that may include an emitter 116 and a detector 118 as described above with respect to FIGS. 1 and 2. As illustrated, the detector 118 may be a detector array that includes a plurality of detector elements 146. The detector array may, for example, be arranged in a one dimensional line or in a two dimensional pattern. The use of a plurality of detector elements 146 may allow for capture of more of the photons emitted by the emitter 116. In this manner, the efficiency of the sensor 114 may be increased. In one embodiment, the emitter 116 and/or the detector 114 may be printed directly onto a flexible substrate 148. The flexible substrate 148 may, for example, be a silicon-based substrate or may be a thermoplastic polymer such as polyethylene terephthalate (PET) foil. Accordingly, the flexible substrate 148 may be a form fitting material that is malleable and maintains its shape once adjusted. In this manner, the flexible substrate 148 may be useful in increasing its tolerance to changing form in response to certain types of motion, such as finger movements, by maintaining a relatively rigid or fixed shape once the sensor has been fitted to the patient. Alternatively, the flexible substrate 148 may be designed to be flexible such that the flexible substrate may maintain contact with a patient 117 as the patient 117 moves. For example, the flexible substrate 148 may be implemented as part of a neonatal forehead probe and as such, the flexible substrate 148 may remain flexible in response to movements of the patient 117.

As described above, the flexible substrate 148 may be part of the sensor 114. As such, the flexible substrate 148 may be affixed to a bandage 150 via, for example, an adhesive. The bandage 150 also may include an adhesive or other affixation element that may be used to affix the sensor 114 to a patient 117. Alternatively, the bandage 150 may include, for example, a soft, pliable, low-profile foam material that allows the sensor 114 to remain in place on a patient 117 without the use of adhesives. The bandage 150 may also be flexible, such that any change in shape of the flexible substrate 148 will be accompanied by a corresponding change in shape of the bandage 150. In one embodiment, the flexible substrate 148 and the bandage 150 may be bent around a center axis 152 such that the emitter 116 is brought into proximity with the detector elements 146. In one embodiment, an extremity of a patient 117, (e.g., an ear, a finger, or a toe) may be placed between the emitter 116 and the detector 118. Thus, the sensor 114 may be bent into shape around a given tissue area of a patient 117, and because of the malleable nature of both the flexible substrate 148 and the bandage 150, the detector array may conform to patient 117 tissue to maximize the light received from the emitter 116 in a manner described in further detail below.

FIG. 4 illustrates the sensor 114 disposed on the tissue of a patient 117 as set forth above. As may be seen, the emitter 116 may, for example, be positioned above the detector elements 146A-N of the detector 118 such that light may pass through the patient 117 via one or more light paths 154. As described above, the emitter 116 may include one or more light emitting diodes (LEDs) that may be used to measure, for example, oxygen saturation, water fractions, hematocrit, or other physiologic parameters of the patient 117. While these detector elements 146A-N are illustrated in a single line, it should be noted that these elements 146A-N may, for example, be arranged in a two dimensional array. In operation, light enters the detector elements 146A-N after passing through the tissue of the patient 117 via light paths 154. The detector elements 146A-N may convert the light at a given intensity, which may be directly related to the absorbance and/or reflectance of light in the tissue of the patient 117, into an electrical signal.

However, there may be bone 156, or other constituents, in the tissue of the patient 117 that may undesirably absorb and/or scatter light from the emitter 116. In this example, the bone 156 may operate to absorb light along given light paths 154 such that given detector elements 146F-I may not receive sufficient light to generate an electrical signal that may be used to calculate the physiologic parameters of the patient 117. However, light may be received at other locations, for example at locations 146B-D and 146J-K) which may be used by, for example, the processor 122 to calculate the physiologic parameters of the patient 117.

Other processing of the signals received at the detector 118 may include the determination of which received signals from a location, such as location 146B, 146C, or 146K, should be used to calculate physiological parameters of the patient 117. As described above, light received at certain locations, such as location 158, may be too weak to properly generate a useable signal for calculation of physiological parameters of the patient 117. Accordingly, the processor 122 may be used to scan the photodetector array in the detector 118 to determine which individual detector elements 146A-N are receiving the strongest light transmission from the emitter 116. The one or more detector elements 146A-N receiving the strongest light transmissions may then be chosen and signals received from the chosen detector elements 146A-N may then be utilized to calculate physiological parameters of a patient 117. In this manner, alternate light paths 154 are available to calculate physiological parameters of a patient 117 instead of only a single light path that might otherwise be unusable due to interference. Thus, the proper operation of the sensor 114 may be improved.

The scan of the detector elements 146A-N outlined above may be performed either continuously or intermittently. In this manner, the processor 122 may be able to take into account changing conditions of the sensor 114 in real time during calculation of physiological parameters of a patient. That is, the processor may factor in changing conditions of the sensor 114 while processing data received from the sensor 114 without any intentional delays being added to the time required to perform the processing, i.e., in real time. For example, if a portion of the detector elements 146A-N previously determined to receive the strongest light transmission from the emitter 116 are exposed to ambient light due to, for example, the bandage 150 becoming loose through movement of the patient 117, the processor 122 may determine that certain detector elements 146A-N have been corrupted in their ability to receive light from the emitter 116. Accordingly, the processor 122 may utilize different detector elements 146A-N for the calculation of physiologic parameters of the patient 117. Thus, the detector elements 146A-N may be scanned in real time so that the best available received light may consistently be selected by the processor 122.

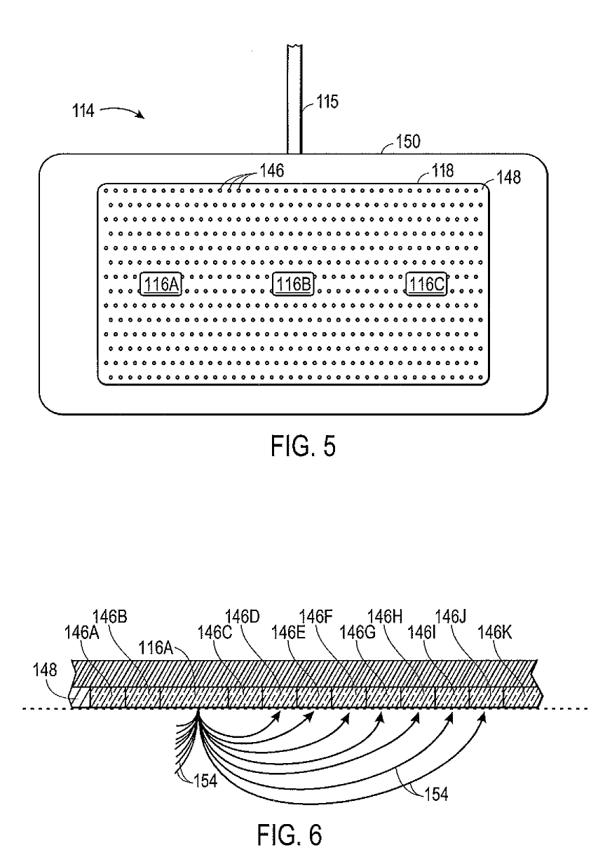

FIG. 5 illustrates a sensor 114 that may utilize a reflectance method to receive light signals. Accordingly, the sensor 114 may include one or more emitters 116, such as three emitters 116A, B, and C, positioned adjacent to the detector elements 146 on the same side of the tissue of a patient 117. Similar to the transmittance type sensor 114 of FIGS. 3 and 4 described above, the sensor 114 of FIG. 5 may include a cable 115 for transmission of signals to and from the sensor 114. The detector elements 146 may) for example, surround the emitters 116. The emitters 116 and/or the detector 114 may be printed directly onto a flexible substrate 148 that may be a silicon based substrate or may be a thermoplastic polymer such as polyethylene terephthalate (PET) foil.

As described above, the flexible substrate 148 may be part of the sensor 114. As such, the flexible substrate 148 may be affixed to a bandage 150 via, for example, an adhesive. The bandage 150 also may include an adhesive or other affixation element that may be used to affix the sensor 114 to a patient 117. In one embodiment, the sensor may be placed on a patient 117, (e.g., on the forehead or finger). The flexible substrate 148 and bandage 150 may be bent into shape around a given tissue area of a patient 117, and because of the nature of both the flexible substrate 148 and the bandage 150, the detector 118 may conform to patient 117 tissue to maximize the light received from the emitters 116.

Furthermore, the use of multiple emitters 116 may be advantageous for the overall efficiency of the sensor 114 through measuring multiple physiological concurrently. For example, if the sensor 114 includes three emitters 116A-C, each of the emitters 116A-C may each transmit light at a different wavelength to the patient 117. Thus the first emitter 116A may transmit light of a given wavelength, such as light in the red spectrum around 660 nm and or light in the infrared spectrum around 900 nm, for determination of the blood oxygen saturation of the patient 117. Additionally, a second emitter 116B may be utilized to determine glucose levels of a patient 117 by transmitting light at a wavelength of approximately 1000 nm. A third emitter 116C may be used to determine hematocrit levels of a patient 117 by transmitting light at a wavelength of approximately 550 nm, Thus, the processor 122 may scan distinct regions near to each of these emitters to receive data relating to multiple tests on a patient 117 simultaneously. Furthermore, the scanning procedure outlined above may be performed for each individual region, such that the strongest signal corresponding to the blood oxygen saturation, glucose level, and hematocrit levels of the patient 117 are being selected.

In another embodiment, the use of multiple emitters 116 may be useful for patients 117 with darkly pigmented skin, because the light is absorbed more completely by the tissue of the patient 117, thus leading to weak signals received at the detector elements 146. Accordingly, to overcome this potential issue, if the detector element 146 scan reveals that all detector elements 146 are receiving weak signals, then the processor 122 may initiate a process whereby two or more adjacent emitters 116A-C may be activated simultaneously to transmit light, for example, at identical wavelengths. In this manner, higher levels of light are transmitted into the patient 117, which may allow, for example, detector elements 146 located between the simultaneously activated emitters 116A-C to receive adequate light for the generation of signals that may be utilized in the calculation of physiologic parameters of the patient 117. Additionally, other efficiencies with respect to the sensor 114 may be obtained, as described below with respect to FIG. 6.

FIG. 6 illustrates a portion of the sensor 114 of FIG. 5 in contact with the tissue of a patient 117. As may be seen, the emitter 116A may, for example, be positioned adjacent to the detector elements 146A-K such that light may pass through the patient 117 via one or more light paths 154. The light paths 154 may, for example, begin at the emitter 116A and end at detector elements 146 D-J, respectively. Accordingly, the light path 154 ending at location 146D is shorter than the light path 154 ending at location 146G, which is shorter than the light path 154 ending at location 146J. Additionally, the light path 154 ending at location 146D is shallower than the light path 154 ending at location 146G, which is shallower than the light path 154 ending at location 146J. Having light paths 154 that pass at different depths and lengths may be advantageous for scanning and selecting signals from detector elements 146 at certain locations 164, 166, or 168. That is, as described above, if, for example, bone or other tissue interferes with the light path 154 to a given location, e.g., 146D, such that a given detector element 146D may not receive sufficient light to generate an electrical signal that may be used to calculate the physiologic parameters of the patient 117, the processor 122 may scan for light received at other locations, for example at locations 146G and/or 146J, which may be used by the processor 122 to calculate the physiologic parameters of the patient 117.

Additionally, the sensor 114 may be utilized to determine physiological parameters for both adults and infants. Adults tend to have thicker skin than infants. Accordingly, light paths 154 typically should go deeper into the skin of an adult patient 117 to properly determine the physiological parameters of the adult patient 117 (e.g., to locations 146G and/or 146J) than the light paths utilized to calculate the physiological parameters of the infant patient 117 (e.g., to location 146D). By having a plurality of detector elements 146A-K, the processor 122 may scan for the best detector element 146 A-F for use with either an adult or an infant patient 117. In this manner, the same sensor 114 may be utilized for both adult and infant patients 117.

While the disclosure may be susceptible to various modifications and alternative forms, specific embodiments have been shown by way of example in the drawings and have been described in detail herein. However, it should be understood that the embodiments provided herein are not intended to be limited to the particular forms disclosed. Indeed, the disclosed embodiments may not only be applied to measurements of blood oxygen saturation, but these techniques may also be utilized for the measurement and/or analysis of other blood constituents. For example, using the same, different, or additional wavelengths, the present techniques may be utilized for the measurement and/or analysis of carboxyhemoglobin, met-hemoglobin, total hemoglobin, fractional hemoglobin, intravascular dyes, and/or water content. Rather, the various embodiments may cover all modifications, equivalents, and alternatives falling within the spirit and scope of the disclosure as defined by the following appended claims.

What is claimed is:

1. A physiological sensor comprising:
an emitter adapted to transmit light;
a detector array comprising a plurality of detector elements each configured to receive the transmitted light via a respective light path; and
a flexible substrate comprising both the emitter and the detector array.

2. The physiological sensor, as set forth in claim 1, comprising a second emitter configured to transmit light at a wavelength different from a wavelength of the emitter.

3. The physiological sensor, as set forth in claim 2, wherein the plurality of detector elements are arranged around the emitter and the second emitter and wherein the plurality of detector elements are configured to receive the transmitted light from each of the emitter and the second emitter.

4. The physiological sensor, as set forth in claim 1, wherein the flexible substrate comprises a material capable of maintaining its shape once adjusted.

5. The physiological sensor, as set forth in claim 4, wherein the material comprises a thermoplastic polymer.

6. The physiological sensor, as set forth in claim 1, wherein the detector elements are organized into a line or into a two dimensional grid.

7. A pulse oximetiy system comprising:
a pulse oximetry monitor; and
a sensor assembly configured to be coupled to the monitor, the sensor assembly comprising:
an emitter configured to transmit light;
an array of detector elements each configured to receive the transmitted light from the emitter; and
a flexible substrate comprising both the emitter and the array of detector elements.

8. The pulse oximetry system, as set forth in claim 7, wherein each of the detector elements is configured to transmit an electrical signal to the pulse oximetry sensor based on the light received from the light emitter.

9. The pulse oximetry system, as set forth in claim 8, comprising a processor configured to scan each of the detector elements for the electrical signal corresponding to the strongest light transmission received from the emitter.

10. The pulse oximetry system, as set forth in claim 9, wherein the processor is configured to select in real time the electrical signal corresponding to the strongest light transmission received from the emitter for calculation of physiological parameters.

11. The pulse oximetry system, as set forth in claim 7, comprising a second emitter configured to transmit light at a wavelength different from a wavelength of the emitter.

12. The pulse oximetry system, as set forth in claim 11, comprising a processor configured to determine a first physiologic parameter based on the light transmitted from the emitter and a second physiologic parameter based on the light transmitted from the second emitter.

13. The pulse oximetry system, as set forth in claim 7, comprising a second emitter configured to transmit light at an identical wavelength to the light transmitted from the emitter, wherein the plurality of detector elements are configured to receive the light from the emitter and the second emitter.

14. A method comprising:
transmitting light via a light emitter located on a flexible substrate;
receiving the light at a light detector element of a light detector array located on the flexible substrate; and
calculating a physiological parameter based on the received light.

15. The method of claim 14, wherein receiving the light at the light detector element on the flexible substrate comprises receiving the light at a plurality of light detector elements surrounding the light emitter.

16. The method of claim 15, comprising generating electrical signals corresponding to the light received at the light detector element of the light detector array and to the light received at a second light detector element of the light detector array.

17. The method of claim 16, comprising scanning the light detector elements to determine the strongest signal and calculating physiological parameters based on the determination.

18. The method of claim 14, comprising generating second light via a second emitter on the on the flexible substrate, wherein the second light comprises light at a wavelength different from a wavelength of the first light.

19. The method of claim 18, comprising calculating a second physiologic parameter based on the second light generated from the second emitter.

20. The method of claim 14, comprising generating second light at a wavelength identical to a wavelength of the first light via a second emitter on the on the flexible substrate, wherein calculating the physiological parameter based on the received light comprises calculating the physiological parameter based on the light generated from the emitter and the second light generated from the second emitter.
